(19) United States
(12) Patent Application Publication
(10) Pub. No.: US 2011/0124681 A1
(43) Pub. Date: May 26, 2011

(54) TOPICAL AND INJECTABLE FORMULATIONS COMPRISING LEUKOTRIENE RECEPTOR ANTAGONIST AND USES THEREOF

(76) Inventor: Larry S. Schlesinger, Maui, HI (US)

(21) Appl. No.: 12/991,671

(22) PCT Filed: May 6, 2009
(86) PCT No.: PCT/US09/42948

§ 371 Date: Dec. 22, 2010

(30) Foreign Application Priority Data
May 6, 2008 (US) 61/050677

(51) Int. Cl. A61K 31/404; A61K 31/47; A61K 31/41; A61K 31/381; A61K 31/352; A61P 17/00; A61P 17/14; A61P 17/02; A61K 8/49; A61Q 19/08

(52) U.S. Cl. 514/311; 514/382; 514/415; 514/443; 514/381; 514/456

(57) ABSTRACT

Methods of treating, preventing, reducing the occurrence of, or slowing progression of folliculitis, partial or full hair loss, thinning of the hair, changes in the texture of hair, graying or whitening (loss of pigmentation) of the hair, dermatological conditions, and other hair-related conditions, comprising administering topical and injectable formulations containing one or more leukotriene receptor antagonists.

### FIELD OF THE INVENTION

The present invention is related to topical and injectable formulations containing one or more leukotriene receptor antagonists and the use thereof to treat and prevent folliculitis, partial or full hair loss, thinning of the hair, changes in the texture of hair, graying or whitening (loss of pigmentation) of the hair, dermatological conditions, and other hair-related conditions.

### BACKGROUND OF THE INVENTION

Hair-related conditions commonly affect both men and women, especially as they age. Folliculitis, partial or full hair loss, hair thinning, changes in the texture of hair, and graying or whitening (loss of pigmentation) of the hair can occur on the head or anywhere on the body. Hair loss, thinning of the hair, changes in the texture of hair, and graying or whitening of the hair, often occur naturally as people age. However, other factors can cause these conditions, such as genetic disposition, infections (bacterial, fungal, and viral), autoimmune disorders, diseases, side effects from medication, trauma, and dermatological conditions of unknown etiology.

Folliculitis is a condition characterized by inflammation and/or infection of the hair follicles. Folliculitis can be characterized by the presence of “ingrown hairs.” Folliculitis can occur after damage occurs to the hair follicles, and it is typically caused by bacterial or fungal infections.

Hair loss and hair thinning typically occur when normal, healthy hairs fall out or become thinner and lose color. Changes in the texture of the hair can also lead to hair loss and hair thinning. The texture of hair can be changed when healthy, normal hairs become terminal or villus hairs. Terminal hairs are hairs that are mature and pigmented but coarse in nature. Villus hairs are hairs that have little color or pigmentation and are generally soft or fine in texture.

Graying or whitening of the hair can be caused by a loss of pigmentation in the hair. Graying or whitening of the hair can also be caused by selective shedding of pigmented hairs.

In addition, some instances of hair loss, hair thinning and graying or whitening of hair are thought to be caused, at least in part, by an inflammatory process. It is thought that inflammation of the hair follicles can cause stress to the follicles, resulting in release of the hair or loss of pigmentation of the hair. Further, scarring of the skin is thought to contribute to hair loss in some cases, as scarring can damage the hair follicles.

Wrinkling of the skin is also a condition experienced by men and women typically as they age. Decreased hormone levels, physical trauma to the skin, and damage caused, for example, by exposure to ultraviolet radiation are among the causes of wrinkling of the skin. Some or all instances of wrinkling of the skin may be affected by inflammatory processes.

Leukotrienes are naturally produced eicosanoid lipid mediators which are involved in allergic and inflammatory responses in the body. Leukotriene antagonists work by inhibiting lipoxygenase and the synthetic pathway of leukotriene and/or blocking leukotriene receptors on target cells. Examples of leukotriene antagonists are acitazanolast, iralukast, montelukast, pranlukast, velukast, zafirlukast, and zileuton.

U.S. Pat. No. 6,762,193 discloses a method of treating inflammatory skin disorders and/or hair loss through oral administration of anti-leukotriene agents, leukotriene synthesis inhibitors, and leukotriene receptor antagonists.

U.S. Pat. No. 6,696,466 discloses a method of treating disorders using leukotriene antagonists. U.S. Pat. No. 6,696,466 discloses inhibiting the graying of scalp hair by inhibiting the progression of the graying process.

There is a need in the art for topical formulations containing leukotriene antagonists for the treatment and prevention of certain hair-related and dermatological conditions.

### SUMMARY OF THE INVENTION

The present invention is directed to a topical and injectable formulation compirising at least one leukotriene antagonist.

The present invention is also directed to a method of treating, preventing, decreasing or reducing the occurrence of, and slowing the progression of hair-related and dermatological conditions, comprising administering an injectable or topical formulation comprising at least one leukotriene antagonist.

The invention is also directed to treating, preventing, reducing the occurrence of, and slowing the progression of folliculitis, hair loss, hair thinning, changes in the texture of hair, graying or whitening (loss of pigmentation) of the hair, and wrinkling of the skin, comprising administering an injectable or topical formulation comprising at least one leukotriene antagonist.

### DETAILED DESCRIPTION OF THE PREFERRED EMBODIMENTS

Leukotriene antagonists useful in accordance with the present invention include, but are not limited to, acitazanolast, iralukast, montelukast, pranlukast, velukast, zafirlukast, and zileuton, or pharmaceutically acceptable salts thereof. Zafirlukast, montelukast, and pranlukast are the most preferred. Leukotriene antagonists include leukotriene receptor antagonists, leukotriene inhibitors, and any agent which blocks the effect of leukotrienes. Of these three compounds, zafirlukast and montelukast are more preferred, and zafirlukast is the most preferred. The term “pharmaceutically acceptable salts” includes salts prepared from pharmaceutically acceptable non-toxic bases or acids including inorganic bases or acids and organic bases or acids.

The term “hair-related conditions” includes, but is not limited to, folliculitis, partial or full hair loss, hair thinning, changes in hair texture, and graying or whitening (loss of pigmentation) of hair. The term “dermatological conditions” includes wrinkling of the skin, eczema, uneven texture of the skin, hyperpigmentation, and scarring caused by conditions such as acne, including acne rosacea, or “adult acne.” The term “hair” refers to any hair on the body, including scalp hair, facial hair, pubic hair, and hair on the arms, chest, and legs.

The administration of an injectable or topical formulation of at least one leukotriene antagonist may result in the treatment, decreased occurrence or slowing of the progression of folliculitis. The administration may result in smoother, evenly colored and evenly textured skin. Preferably, the administration is topical.

The administration of an injectable or topical formulation of at least one leukotriene antagonist may result in the treatment, prevention, reduced occurrence of, or slowing of the progression of hair loss. Preferably, the administration is topical. The administration of an injectable or topical formulation of at least one leukotriene antagonist may result in an increased rate of hair growth. The administration may result in reversal or slowing of the progression of hair loss. The effects of the administration are not limited to the site of injected or topical administration, although preferably the effects occur at or around the site of administration.

The administration of an injectable or topical formulation of at least one leukotriene antagonist may result in an initial period of time where existing hair, including grey, terminal, and/or villous hair, falls out of the follicles and is later replaced by healthy new hairs which may be thicker in texture and have increased texture.

The administration of an injectable or topical formulation of at least one leukotriene antagonist may result in the treatment, prevention, reduced occurrence of, or slowing of the progression of hair thinning. The administration may result in an increase in number of hairs, as well as an increase in the thickness of the hair.

The administration of an injectable or topical formulation of at least one leukotriene antagonist may result in the treatment, prevention, reduced occurrence of, or slowing of the progression of changes in hair texture. The administration may result in the conversion of villus or terminal hairs to normal, healthy scalp hairs.

The administration of an injectable or topical formulation of at least one leukotriene antagonist may result in the treatment, prevention, reduced occurrence of, or slowing of the progression of graying or whitening (loss of pigmentation) of hair. The administration may slow or inhibit the formation of grey or white hairs (hairs with decreased or no pigmentation). The administration, preferably topical, may also cause gray or white hairs (hairs with decreased or no pigmentation) to have increased or full pigmentation.

The administration of an injectable or topical formulation of at least one leukotriene antagonist may result in the initial release of villus hair, terminal hairs, and/or gray or white hairs and subsequent growth of healthy scalp hairs.

The administration of an injectable or topical formulation of at least one leukotriene antagonist may result in the treatment, prevention, reduced occurrence of, or slowing of the wrinkling of skin. The administration may cause “rejuvenation” of the skin, resulting in the decrease of wrinkles in the skin and improved texture and pigmentation of the skin.

Preferably, the method of preventing the occurrence of hair-related conditions and dermatological conditions by administering an injectable or topical composition comprising at least one leukotriene antagonist occurs before the conditions occurs.

Preferably, the methods of treating, reducing the occurrence of, slowing the progression of the hair-related conditions and dermatological conditions comprising administering an injectable or topical composition comprising at least one leukotriene antagonist occur soon after the formation or development of the condition(s). For example, the commencement of administering the an injectable or topical composition comprising at least one leukotriene antagonist occurs preferably within five years, more preferably within one year, and most preferably within one to six months of the occurrence of folliculitis, hair loss, hair thinning, change in hair texture, graying or whitening (loss of pigmentation) of hairs, and wrinkling of the skin.

However, administration of the injectable or topical formulation at any time after the occurrence of the hair-related conditions and dermatological conditions may alleviate or eliminate the condition.

The patient may be administered the injectable or topical formulation indefinitely. It is preferable that the administration occur without a lapse of more than one year, more preferably without a lapse of more than one month, and most preferably without a lapse of more than one week. However, the administration of the injectable or topical formulation need not be continuous. In other words, a patient may be removed from administration and later have the injectable or topical formulation administered again.

Topical dosage forms include any form suitable for topical, e.g., transdermal, application, and include but are not limited to, aerosols, gels, solutions, suspensions, lotions, pastes, creams, ointments, dusting powders, patches, foams, and the like. Of these dosage forms, aerosols, gels, solutions, lotions, creams, and ointments are preferred, and foams are preferred. These administration forms may be presented in unit dosage form and may be prepared by any of the methods well-known in the art of pharmacy. The active agent is typically present in an amount of from 1 to 99% by weight, based upon the total weight of the formulation, for example from 10 to 50% by weight.

Injectable dosage forms include any form suitable for parenteral administration, including but not limited to, intravenous (IV), intramuscular (IM), intradermal, subcutaneous (SC), intracoronary, intraarterially, pericardially, intraarticular, percutaneous, subfascial and intraperitoneal (IP) injections. In preferred embodiments, injectable dosage forms are administered intradermally, percutaneously, subfascially, or subcutaneously. (See, e.g., Robinson et al., 1989, In: Pharmacotherapy: A Pathophysiologic Approach, Ch. 2, pp. 15-34, incorporated herein by reference in its entirety.)

The magnitude of a therapeutic dose varies with the nature of the severity of the condition to be treated and with the particular compound used. The dosage will also vary according to the age, weight and response of the individual patient. In general, the disclosed daily dose range for any use is within the range of from about 0.001 mg to about 100 mg per kg body weight of a mammal, preferably 0.01 mg to about 10 mg per kg, and most preferably 0.1 to 1 mg per kg.

In some preferred embodiments, zafirlukast is used. In preferred embodiments, the total daily dosage of zafirlukast, which may be from a single dose or multiple doses, can be 5 to 500 mg, preferably 10 to 100 mg, more preferably 15 to 75 mg, and most preferably 20 to 50 mg.

In preferred embodiments, the formulation may be administered preferably one to ten times daily, more preferably one to three times daily, and most preferably one time daily.

In preferred embodiments, the administration of the injectable or topical formulation may a “pulse” therapy, where formulation is administered for a specific time period, then discontinued for a time period, and then readministered continuously in an “on and off” fashion. In preferred embodiments, the time period is 30 days.

In some embodiments, the topical formulation of the invention comprises an ointment, which is a semisolid pharmaceutical preparation based on well known materials such as an oleaginous base, lanolin, emulsions, or water-soluble bases. Preparation of ointments is well known in the art. Such preparations may contain petrolatum or zinc oxide together with the active agent. Oleaginous ointment bases suitable for use in the present invention include generally, but are not limited to, vegetable oils, animal fats, and semisolid hydrocarbons obtained from petroleum. Absorbent ointment bases of the present invention may contain little or no water and may include components such as, but not limited to, hydroxystearin sulfate, anhydrous lanolin and hydrophilic petrolatum. Emulsion ointment bases of the present invention are either water-in-oil (W/O) emulsions or oil-in-water (O/W) emulsions, and may include, but are not limited to, cetyl alcohol, glyceryl monostearate, lanolin, polyalkylsiloxanes, and stearic acid. Water-soluble ointment bases suitable for use in the present invention may be prepared from polyethylene glycols of varying molecular weight. In an additional aspect, ointments of the present invention may include additional components such as, but not limited to, additional active agents, excipients, solvents, emulsifiers, chelating agents, surfactants, emollients, permeation enhancers, preservatives, antioxidants, lubricants, pH adjusters, adjuvants, dyes, and perfumes. The specific choice and compositions of such additional components may be made by those skilled in the art in accordance with the principles of the present invention.

In another aspect of the present invention, a cream may be prepared in accordance with the principles of the present invention. Creams are a type of ointment which are viscous liquids or semisolid emulsions, either oil-in-water or water-in-oil, as is well known in the art. Cream bases may be soluble in water, and contain an oil phase, an emulsifier, an aqueous phase, and the active agent. In one embodiment of the present invention, the oil phase may be comprised of petrolatum and a fatty alcohol such as cetyl or stearyl alcohol. In another embodiment of the present invention, the aqueous phase may exceed the oil phase in volume, and may contain a humectant. In another embodiment of the present invention, the emulsifier in a cream formulation may be a nonionic, anionic, cationic or amphoteric surfactant. For an oil-in-water emulsion, the water phase of the cream may contain between about 20 and about 60% w/w of water, between about 1 and about 15% w/w of at least one emulsifier, up to about 50% w/w of an oil phase, and up to about 1% w/w of a preservative such as a paraben. The oil phase of the cream may contain up to about 40% w/w of a solvent, up to about 15% w/w of at least one emulsifier, up to about 40% w/w of an oil phase, and up to about 1% w/w of a preservative such as a paraben.

In another embodiment of the present invention, a lotion may be prepared. A lotion is a composition which may be a liquid or semi-liquid preparation in which solid particles, including the active agent, are present in a water or alcohol base. Lotions suitable for use in the present invention may be a suspension of solids or may be an oil-in-water emulsion. In another embodiment of the present invention, lotions may also contain suspending agents which improve dispersions or other compounds which improve contact of the active agent with the skin, e.g., hydrophilic polymers such as methylcellulose, sodium carboxymethylcellulose, or similar compounds. Lotions of the present invention may include additional components such as, but not limited to, additional active agents, excipients, solvents, emulsifiers, chelating agents, surfactants, emollients, permeation enhancers, preservatives, antioxidants, lubricants, pH adjusters, adjuvants, dyes, and perfumes. The specific choice and compositions of such additional components may be made by those skilled in the art in accordance with the principles of the present invention and may differ from the components which would be chosen for other topical formulations of the present invention.

In another embodiment of the present invention, the lotion may be an emulsion of a water and oil phase. The water phase of the lotion may contain between about 20% w/w to about 90% w/w of an excipient such as water, up to about 5% w/w of a surfactant, up to about 5% w/w of a buffering agent such as sodium chloride or the like, and up to about 1% w/w of a preservative such as a paraben. The oil phase of the lotion may contain up to about 40% w/w of at least one solvent such as glycerin and cetyl alcohol, up to about 10% w/w of an absorbent base such as petrolatum, up to about 5% w/w of an antioxidant such as isopropyl palmitate, up to about 5% w/w of an oil phase such as dimethicone, and up to about 1% w/w of a preservative such as a paraben.

In yet another embodiment of the present invention, a paste may be prepared in accordance with the present invention. Pastes of the present invention are compositions in which there are significant amounts of solids which form a semisolid formulation in which the active agent is suspended in a suitable base. In one embodiment of the present invention, pastes may be formed of bases to produce fatty pastes or made from a single-phase aqueous gel. Fatty pastes suitable for use in the present invention may be formed of a base such as petrolatum, hydrophilic petrolatum or the like. Pastes made from single-phase aqueous gels suitable for use in the present invention may incorporate cellulose based polymers such as carboxymethylcellulose or the like as a base. Pastes of the present invention may include additional components such as, but not limited to, additional active agents, excipients, solvents, emulsifiers, chelating agents, surfactants, emollients, permeation enhancers, preservatives, antioxidants, lubricants, pH adjusters, adjuvants, dyes, and perfumes.

In another embodiment of the present invention, a gel may be prepared. A gel prepared in accordance with the present invention may be a preparation of a colloid in which a disperse phase has combined with a continuous phase to produce a viscous product. The gelling agent may form submicroscopic crystalline particle groups that retain the solvent in the interstices. As will be appreciated by those working in art, gels are semisolid, suspension-type systems. Single-phase gels can contain organic macromolecules distributed substantially uniformly throughout a carrier liquid, which may be aqueous or non-aqueous and may contain an alcohol or oil. A variety of specific gel vehicles are known to those of ordinary skill in the art. Examples of specific gel types, their manufacture and use may be found, for example, in U.S. Pat. Nos. 2,909,462; 4,340,706; 4,652,441; 5,516,808; 5,643,584; 5,840,338; 5,912,009; and 6,258,830, each of which are incorporated herein by reference in their entirety. In some embodiments, the gel formulation may be prepared by providing a gelling agent, usually in a powdered form, and adding an excipient such as water in the case of a hydrophilic gelling agent or mineral oil in the case of a hydrophobic gelling agent. The gel then swells and may be optionally neutralized. In a separate vessel, the active agent may be dissolved in an appropriate solvent. The dissolved active agent and the gel may then be mixed to form the final gel formulation. Other methods of producing a drug-containing gel will be recognized by those of ordinary skill in the art. The gel may include a variety of additional components such as, but not limited to, additional active agents, excipients, solvents, emulsifiers, chelating agents, surfactants, emollients, permeation enhancers, preservatives, antioxidants, lubricants, pH adjusters, adjuvants, dyes, and perfumes. Further, in order to prepare a uniform gel, dispersing agents such as alcohol or glycerin can be added, or the gelling agent can be dispersed by trituration, mechanical mixing or stirring, or combinations thereof. It will be recognized, however, by those skilled in the art that other methods and means of incorporating the active agent and other components into the gel may be employed consistent with the teachings of the present invention.

In one embodiment of the present invention, aqueous gels may comprise water or water/ethanol and about 1-5 wt % of a gelling agent. In another aspect of the present invention, non-aqueous gels may be comprised of silicone fluid, such as colloidal silicon dioxide, or mineral oil. The suitability of a particular gel depends upon the compatibility of its constituents with both the active agent and a permeation enhancer, if used, and any other components in the formulation.

In accordance with the present invention, the gelling agent may be a compound of high molecular weight which acts as a thickening agent to produce a semisolid or suspension-type formulation. Gelling agents may be hydrophobic or hydrophilic and are generally polymers. Gels which incorporate hydrophilic polymers are referred to as hydrogels, as is understood by those skilled in the art. Examples of suitable gelling agents for use in the present invention may include synthetic polymers such as, but not limited to, polyacrylic acids or poly(1-carboxyethylene), carboxypolymethylenes prepared from acrylic acid cross-linked with allyl ethers of (polyalkyl) sucrose or pentaerythritol (e.g. CARBOPOL 940/941/980/981/1342/1382 and carbamer polymers such as carbomer 934P/974P), sodium acrylate polymers (e.g. AQUAKEEP J-550/J-400), other polycarboxylic acids, alkyl acrylate polymers (e.g. PEMULEN), and mixtures or copolymers thereof. In another embodiment of the present invention, suitable gelling agents may include vinyl polymers such as but not limited to carboxyvinyl polymers, polyvinyl pyrrolidone, polyvinyl alcohol, polyvinyl methyl ether, polyvinyl ether, polyvinyl sulfonates, and mixtures or copolymers thereof. In a further embodiment of the present invention, suitable gelling agents may include polymers such as but not limited to polyethylene compounds (e.g. polyethylene glycol, etc.), polysaccharides (e.g. polysucrose, polyglucose, polylactose, etc.) and salts thereof, acrylic acid esters, alkoxybutyninpolymers (e.g. polyoxyethylene-polyoxypropylene copolymers such as the PLURONIC line of BASF, Parsippany, N.J.), polyethylene oxide polymers, polyethers, gelatin succinate, colloidal magnesium aluminum silicate (which may be useful as a gel stabilizer in conjunction with another gelling agent), petroleum jelly and mixtures of copolymers thereof.

Suitable gelling agents also include cellulose polymers such as hydroxypropyl cellulose (e.g. KLUCEL), hydroxypropylmethyl cellulose (e.g. KLUCEL HF, METHOCEL), hydroxypropylethyl cellulose, hydroxypropylbutyl cellulose, hydroxypropylpentyl cellulose, hydroxyethyl cellulose (NATROSOL), ethylcellulose, carboxymethyl cellulose, hydroxypropylmethyl cellulose phthalate, and cellulose acetate. Suitable gelling agents may also be natural gelling agents include, dextran, gaur-gum, tragacanth, xanthan gum, sodium alginate, sodium pectinate, sodium alginate, acacia gum, Irish moss, karaya gum, guaiac gum, locust bean gum, etc., while natural high molecular weight compounds include, among others, various proteins such as casein, gelatin, collagen, albumin (e.g. human serum albumin), globulin, fibrin, etc. and various carbohydrates such as cellulose, dextrin, pectin, starches, agar, mannan, and the like. These substances may be also be chemically modified, e.g. esterified or etherified forms, hydrolyzed forms (e.g. sodium alginate, sodium pectinate, etc.) or salts thereof.

The amount of gelling agent employed in a gel of the present invention may vary depending on the specific result to be achieved. However, in one aspect, the amount of gelling agent may be from about 0.05 to about 10 wt % of the gel formulation. In a more preferred aspect, the amount of gelling agent may be 0.1 to 5 wt % of the gel formulation prior to introduction of the active agent and any accompanying components.

In some embodiments of the present invention, an emulsifier may be used, preferably when a solvent is used. Emulsifiers suitable for use in the present invention include, but are not limited to, polyols and esters thereof such as glycols, propylene glycol, polyethylene glycol, glycolhexylene glycol, ethylene glycol, glycerol, butanediol, polyethylene glycol monolaurate, and propylene glycol ester of alginic acid. Emulsification may be accomplished by conventional dispersion techniques. For example, intermittent shaking, mixing by means of a propeller mixer, turbine mixer or the like, colloid mill operation, mechanical homogenization, ultrasonication, or other known methods may be utilized. Emulsifiers may form stable oil-in-water emulsion, and such emulsifiers are exemplified by anionic surfactants (e.g. sodium oleate, sodium stearate, sodium laurylsulfate, etc.), nonionic surfactants (e.g. polyoxyethylene sorbitan fatty acid esters (Tween 80 and Tween 60, Atlas Powder, U.S.A.), polyoxyethylene castor oil derivatives (HCO-60 and HCO-50, Nikko Chemicals, Japan], etc.), polyvinyl pyrrolidone, polyvinyl alcohol, carboxymethylcellulose, lecithin, gelatin, and combinations thereof. The concentration of the emulsifier may be selected from the range of about 0.01% to about 20%. It will be noted that many of these emulsifiers also act as gelling agents.

Solvents or solubilizing agents may also be used in the topical composition. Suitable solvents for use in the present invention include, but are not limited to lower alcohols, ethanol, isopropanol, benzyl alcohol, propanol, methanol, other C₄-C₁₀ mono-alcohols and mixtures thereof. In another aspect the solvents suitable for use in the present invention may include albumin, gelatin, citric acid, ethylenediamine sodium tetraacetate, dextrin, dimethylsulfoxide, dimethylacetamide, dimethylformamide, 2-pyrrolidone, N-(2-hydroxyethyl) pyrrolidone, N-methylpyrrolidone, 1-dodecylazacycloheptan-2-one and other n-substituted-alkyl-azacycloalkyl-2-ones (azones), sodium hydrosulfite and mixtures thereof. In some embodiments, the topical formulation of the invention comprises a polar aprotic solvent, preferably selected from any one or more of the following: dimethylsulfoxide, dimethylacetamide, dimethylformamide or N-methylpyrrolidone. Dimethylsulfoxide is most preferred. The amount of the solvent in the topical formulation is preferably about 25-90% by weight, more preferably about 50-80% by weight, based on the total weight of the excipients in the formulation (i.e., not including the active agent(s)).

In some embodiments, the topical formulation of the invention comprises one or more carbamides. The one or more carbamides may include urea, carbamide peroxide, urea-D glucuronic acid, allantinon (5-ureidohydantoin), urea phosphate, urea sulfate, ureidoglycolic acid (glyoxylurea), ureidopropionic acid (N-Carbamyl-Balanine), ureidosuccinic acid (N-Carbamyl-aspartic acid), N-Carbamyl-arginine, N-carbamylglycine (hydantoic acid), N-carbamyl-phenylalanine or glycolylurea (hydantoin). Urea is the most preferred. The one or more carbamides may be present in amounts of about 5-50% by weight, more preferably about 10-40% by weight.

In some embodiments, the topical formulation of the invention comprises water in amounts of about 1-25% by weight, more preferably about 2-10% by weight.

In some embodiments, the injectable formulation is in a sterile injectable form, such as a solution, suspension or emulsion. The injectable formulation is preferably isotonic with the blood of the recipient with a pharmaceutically acceptable carrier. Examples of such sterile injectable forms are sterile injectable aqueous or oleaginous suspensions. These suspensions may be formulated according to techniques known in the art using suitable dispersing or wetting agents and suspending agents. The sterile injectable forms may also be sterile injectable solutions or suspensions in non-toxic parenterally-acceptable diluents or solvents. Among the acceptable vehicles and solvents that may be employed are water, saline, Ringer's solution, dextrose solution, isotonic sodium chloride solution, and Hanks' solution, or mixtures thereof. In addition, sterile, fixed oils are conventionally employed as solvents or suspending mediums. For this purpose, any fixed oil may be employed including synthetic mono- or di-glycerides, corn, cottonseed, peanut, and sesame oil. Fatty acids such as ethyl oleate, isopropyl myristate, and oleic acid and its glyceride derivatives, including olive oil and castor oil, especially in their polyoxyethylated versions, are useful in the preparation of injectables. These oil solutions or suspensions may also contain long-chain alcohol diluents or dispersants.

In the compositions suitable for percutaneous administration, the carrier may optionally comprise a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions.

Unless explicitly stated differently, all weight percentages herein are based on the total weight of the excipients in the formulation (i.e., not including the active agent(s)).

### EXAMPLE

TOPICAL FORMULATION: 1 mg/mL zafirlukast

800 mg zafirlukast

220 mL propylene glycol

340 mL of 100% ethanol

120 mL water for irrigation

Quantity sufficient to 800 mL with glycerin (approximately 105 mL)

1. A method of treating, preventing, reducing the occurrence of, or slowing the progress of hair-related and dermatological conditions in a patient in need thereof, comprising administering an injectable or topical formulation comprising at least one leukotriene antagonist.

2. The method of claim 1, wherein the leukotriene receptor antagonist is selected from the group consisting of acitazanolast, iralukast, montelukast, pranlukast, velukast, zafirlukast, zileuton, and pharmaceutically-acceptable salts thereof.

3. The method of claim 1, wherein the formulation is a topical formulation comprising zafirlukast.

4. The method of claim 3, comprising administering zafirlukast in a total daily dosage of 5 to 500 mg.

5. The method of claim 4, wherein the total daily dosage of zafirlukast is 20 to 50 mg.

6. The method of claim 1, wherein the formulation is administered one to ten times daily.

7. The method of claim 6, wherein the formulation is administered one time daily.

8. The method of claim 1, wherein the hair related condition is selected from the group consisting of: folliculitis, hair loss, hair thinning, changes in hair texture, and loss of pigmentation of the hair.

9. The method of claim 1, wherein the dermatological condition is selected from the group consisting of: wrinkling of the skin, eczema, uneven texture of the skin, hyperpigmentation, and scarring.

10. The method of claim 1, wherein the topical formulation is a dosage form selected from the group consisting of: aerosols, gels, solutions, suspensions, lotions, pastes, creams, ointments, dusting powders, foams, and patches.

11. The method of claim 1, comprising administering the injectable formulation intravenously, intradermally, percutaneously, subfascially, or subcutaneously.
